(19) United States
(12) Patent Application Publication
(10) Pub. No.: US 2021/0022903 A1
(43) Pub. Date: Jan. 28, 2021

(54) NASAL AIR FLOW MAINTENANCE AND SEPTAL DEVIATION CORRECTION DEVICE

(71) Applicant: Sabujan Sainudeen, Kollam (IN)

(72) Inventor: Sabujan Sainudeen, Kollam (IN)

(21) Appl. No.: 16/521,705

(22) Filed: Jul. 25, 2019

(51) Int. Cl. A61F 5/08; A61F 5/56

(52) U.S. Cl. CPC A61F 5/08; A61F 5/56

(57) ABSTRACT

A first prong and a similarly configured second prong are each integrally formed with an upper section, a lower section, and an intermediate section there between. Each upper section is linear and of an extended length. Each lower section is arcuate and of a shortened length thereby forming first and second handles. A spring is positioned between the lower sections of the prongs and is coupled to the first and second handles. A first vent is coupled to the upper section of the first prong. A similarly configured second vent is coupled to the upper section of the second prong. Each vent has a smaller configuration above and a larger configuration below.

### BACKGROUND OF THE INVENTION

### Field of the Invention

The present utility model and design relates to a hygienic, safe, self use device which can be used for the purpose of improving nasal air flow during breathing. It is meant for safe and effective widening of anterior nasal pathway in persons with congested, roomy or collapsing nasal passage during nasal breathing. More importantly, it is a nonsurgical alternative for those suffering from deviation of nasal septum. It can also give effective relief in persons from snoring and obstructive sleep apnea secondary to or associated with nasal obstruction. Obstruction of anterior nasal pathway is a common problem in those with allergies, congested nasal mucosa and with severe deviation of nasal septum. This can produce reduction in exercise tolerance, athletic performance and a common cause for snoring, obstructed sleep apnea and related complications. This invention features a safe, self-introducible device into anterior nasal cavity which/widens up anterior nasal chambers, corrects cartilaginous cause of deviated nasal septum and prevent collapse of nasal pathway as occurs during deep breathing or during sleep which can cause uneasiness, poor athletic performance, snoring, reduce quality of sleep and poor post sleep performance. Also the system is more specifically designed as a small barely noticeable device with good safety features as needed during sleep and non-contact sports. Also, allowing a wide range of movement of head in bed along with the device, unlike other alternatives.

### Description of the Prior Art

Nasal obstruction or feeling of nasal stuffiness is a common problem of general population. There are various causes of nasal obstruction including deviated nasal septum, stuffy nasal mucosa, collapse of anterior nasal cavity during forced breathing or sleep. Various devices are developed and patented for maintaining anterior nasal opening and air passage. These are generally plastic devices with different shapes and sizes which can be introduced into nostrils maintaining the orifice. Some of the devices which can be stickered over nostrils and resultant stretching may prevent collapse of nasal passage. But the anterior nasal passages are mostly bound by cartilaginous frame covered by a mucosa and are reasonably rigid for many soft plastic devices to correct these malformations. Also these devices are meant to make only the initial one centimeters, or so of nasal passage. More often the cause of nasal blockage is more widespread and involves deep nasal passage on which these devices are ineffective. Moreover, the current existing devices are not suitable for deviation of nasal septum resulting from unusual bend in the nasal septal cartilage, which are common causes of persistent nasal blockage.

In this regard a simple self-introducible device which can dilate the nostrils, widens the anterior nasal cavity and correct deviation of nasal septum may be a valuable addition to the existing devices used to improve nasal air flow. Also the present invention offers a better and effective alternative to existing devices allowing firm grip which maintain it in position. In this regard, the current invention incorporates all the advantages of different nasal cavity devices with none of the disadvantages.

The use of different models of intra nasal air flow improvement devices are known in the prior art. More specifically, such methods, systems, and devices previously devised and utilized for the various purpose are known to consist basically of familiar, expected, and obvious structural configurations, notwithstanding the myriad of designs encompassed by the crowded prior art which has been developed for the fulfillment of countless objectives and countless requirements. While these systems and devices fulfill their respective, particular objectives and requirements, the aforementioned methods, systems, and devices do not describe the nasal air flow maintenance and septal deviation correction device that allow safe, effective improvement in nasal air flow during rest, exercise or sleep.

In this respect present invention substantially departs from the conventional concepts and designs of the prior art, and in doing so provide an apparatus primarily developed for the purpose of ergonomically widening the nasal pathway from roomy nasal cavities, stuffy nose, deviation of nasal septum or collapsing nasal passage during forced inspiration or sleep. Therefore, it can be appreciated that there exists a continuing need for a new and improved nasal air flow device, which can be used at any place or time with minimal discomfort and/or harm. In this regard, the present invention substantially fulfills this and various other needs.

### SUMMARY OF THE INVENTION

In view of the disadvantages inherent in the known types of nasal air flow improvement devices now present in the prior art, the present invention provides an improved nasal septal deviation correction device and air flow facilitator. As such, the general purpose of the present invention, which will be described subsequently in greater detail, is to provide improved breathing, improve comfort and performance which has all the advantages of the prior art and none of the disadvantages. In this respect, before explaining at least one embodiment of the invention in detail, it is to be understood that the invention is not limited in its application to the details of technique and to the designs of the components and method steps set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting. As such, those skilled in the art will appreciate that the conception, upon which this disclosure is based, may readily be utilized as a basis for the designing of other structures, methods, and systems for carrying out the several purposes of the present invention including animal (veterinary) versions and medicament incorporated devices. It is important, therefore, that the invention be regarded as including such equivalent constructions insofar as they do not depart from the spirit and scope of the present invention. For a better understanding of the invention, its operating advantages and the specific objects attained by its uses, reference should be given to the accompanying drawings and descriptive matter in which there is illustrated a primary, a preferred embodiment of the invention and alternate embodiments of the invention.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims attached.

For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood For better understanding the concept and presenting the various modifications of the details and different embodiments, the invention is described in two steps. One is primary structural design of current invention and secondly a detailed description of preferred embodiment with structural and ornamental or decorative details. The invention will be better understood and objects other than those set forth above will become apparent when consideration is given to the following detailed description thereof. Such description makes reference to the annexed drawings wherein:

FIG. 1 is a top elevational schematic view of the primary embodiment of a nasal air flow maintenance and septal deviation correction device constructed in accordance with the basic principles of the present invention. It shows relative position of structural components while the device is idle and when positioned inside the nasal cavities.

FIG. 2 is a plan view of the system as in FIG. 1 with the head switch pressed for widening the prongs for introduction of device and for withdrawing after placing inside the nasal cavities.

FIG. 3 is a magnified schematic top elevational view of the head assembly of the mechanical part of the present invention.

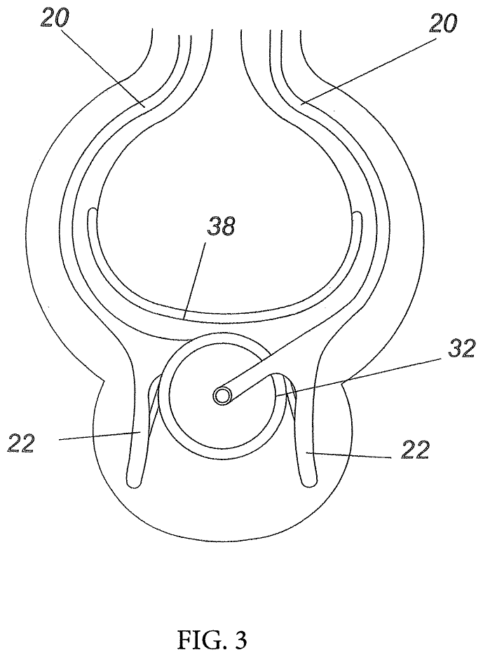

FIG. 4 is a top elevational plan view of the preferred embodiment of the nasal air flow maintenance and septal deviation correction device of the present invention.

FIG. 5 is a plan view of the alternative embodiment of nasal air flow maintenance and septal deviation correction device of the present invention.

The same reference numerals refer to the same parts throughout the various figures.

### DESCRIPTION OF THE PREFERRED EMBODIMENT

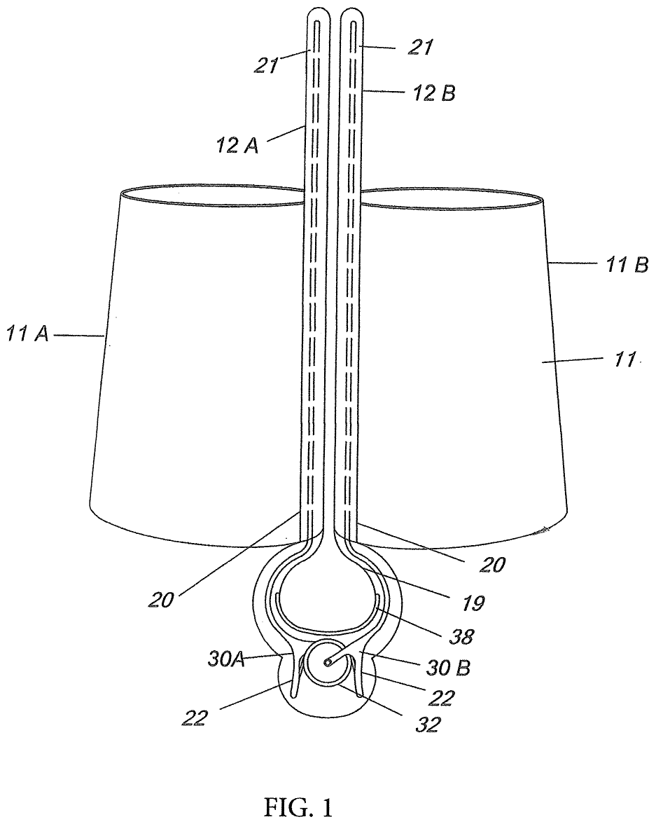

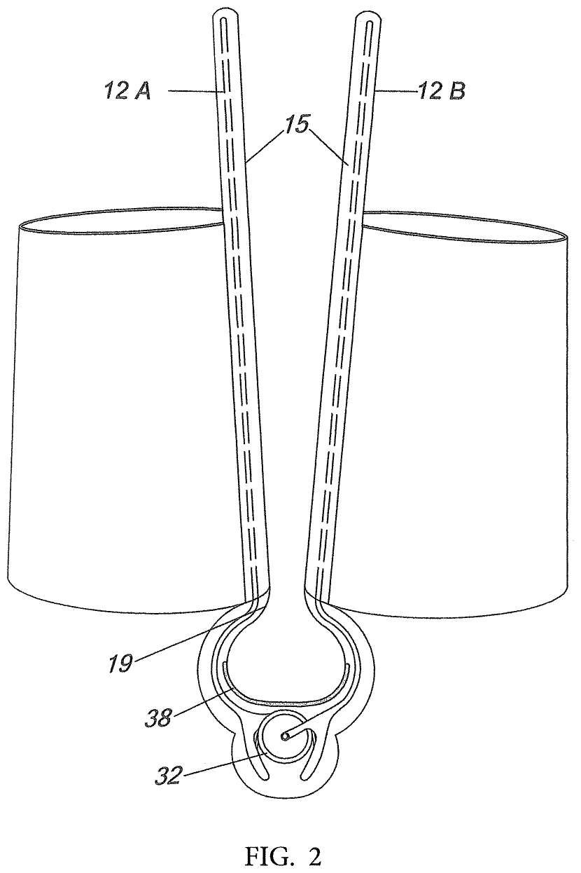

With reference now to the drawings, and in particular to FIGS. 1 and 2 thereof, the fundamental concept of the new and improved nasal air flow maintenance and deviation correction device embodying the principles and concepts of the present invention and generally designated by the reference numeral 10 will be described.

The present invention is a modified nasal air flow maintenance and septal deviation correction device. It is comprised of a plurality of components. Such components are individually configured and correlated with respect to each other so as to attain the desired objectives. It is to be mentioned here that the description of embodiments of the invention described herein, any reference to orientation or direction are merely intended for convenience of description and is not intended in any way to limit the scope of the present invention. Relative terminologies used such as “above,” “below,” “front,” “rear,” “up,” “down,” “lower,” “upper,” “horizontal,” “vertical,” “anterior,” “posterior,” as well as derivatives thereof, e.g., “downwardly,” “upwardly,” “horizontally,” etc., should be construed to refer to the orientation as then described or as shown in the drawing under discussion. These relative terms are merely meant for convenience of description and do not require that the apparatus be constructed or operated in a particular orientation unless explicitly indicated as such. Terms such as “coupled”, “fabricated”, “connected,” “attached,” and similar refer to a relationship of structures which are secured or attached to one another either directly or indirectly.

In their broadest context, the primary embodiment of the invention is a modified nasal air flow maintaining device 10 which includes a pair of nasal air flow maintenance vents 11A, 11B, deviation correction prongs which form a composite unit integrated by a handle combined with pressure opener.

An air flow vent is a soft cylindrical vent which forms one of the major components of the device. The air flow vents are smooth polymeric or silicon tubes which fill inside the nasal cavities. Each vent is flexible and smooth to be inserted safely inside a nasal cavity. It is reasonably rigid to maintain the nasal cavity caliber and prevent the anterior nasal cavity from collapsing during breathing and stretches out a roomy nasal cavity by its elastic recoiling nature. Since it fills inside the cavity and is reasonably rigid, air can flow through the central cylindrical hole without being obstructed. Each air flow vent has an inside opening and an anterior or forward opening close to the external orifice of nasal cavity.

Two rigid, flat cuboid like central septal prongs are attached on the inner or medial surface of the air flow vent. The central septal prongs are three dimensional structures with smooth surfaces and blunt ends on the posterior or rear ends. The central septal prongs are rigid structures inserted on each of the two nasal cavities and act as the septal deviation correction component and principal holder for the pair of air vents. Central septal prongs are metallic, ideally stainless steel or other rigid metal sheets covered by a polymeric smooth coating to avoid trauma to delicate mucosal surfaces of nasal cavity. The central septal prongs are long structures which are longer than the distal end of air vents forming as extra projections beyond the air vents. This is to correct the long nasal septum as long as reasonably possible in a safe manner. The two nasal septal prongs are parallel structures, one for each of the inner side of nasal cavities. They are held firmly together to exert pressure on central nasal septum to straighten it.

The air vents are approximately 15+/−5 mm long and a caliber of 12+/−5 mm. The shell thickness is kept as minimum as possible, but to retain sufficient strength of toughness and elasticity to maintain the shape against usual resistance inside the nasal cavity and may be about 0.5+/−0.3 mm. The nasal septal prongs when measured from the outer margin of air vents are about 20+/−5 mm long and are 1+/−0.5 mm thick and 10+/−5 mm wide, an upper to lower surface distance.

The nasal septal prongs with attached air vents on their inner sides are held together at a handle situated at the front end. The handle is basically a curved extension of the nasal septal prongs along with their soft coating on surfaces. The handles of the device are semispherical as the proximal ends of metal prongs meet forming a curved ending. These curved endings cross each other and are held together by a metallic coil spring. The metallic coil spring is tight so as to exert good negative force to maintain the nasal septal prongs closely together and to provide sufficient strength to tighten the nasal septum correcting any curvature defect called “deviation of nasal septum.

The two nasal septal prongs and air vents are normally kept parallel and tightly together by the negative pressure from metallic coil spring. This also holds the device securely inside the nasal cavity without loosening. This is a departure from the usual existing air vent alone models. The central area of the device has a parallel gap to accommodate the nasal septum. The handle assembly is widened so as to accommodate the anterior extension of nasal septum, nasal columella which it is wider and is securely protected by the wide gap at the center and more curved structure around this area.

The handle assembly has a semi dumbbell shaped outer configuration with a soft covered area over the handle anteriorly which is the forward end of device. This area when pressed exerts positive pressure on metal coil spring so as to open up the nasal septal prong pairs and widen the gap between the two along with the attached air vents. This is needed to remove the device from nasal cavity and to securely place the device inside.

The nasal septal prongs are a pair of flat cuboidal structures with a metal core and outer smooth soft elastomeric coating. Each prong has an outer surface and inner surface. The main component of nasal septal prongs is the flat metal which is a rigid and strong material with minimal malleability such as tempered stainless steel or other metals, alloys or rigid plastic material. One is an open end, central section/semi-circle, with blunt margins projecting outward, and forms the handles 30a, 30b which can be pressed for opening the device. The other end courses medially and comes to contact with its pair and joined together by a metal axle cum stopper 31. A wheel shaped metallic spring separates the pair and secures them in place and are joined with each other by inserting the ends of metal spring ends, into the handles. Thus these nasal septal prongs have a handle assembly area and a long intra nasal upper area with a curved bend at the expected area of outer orifice of nasal cavity. The tips, the distal ends of the prongs, are of metal, curved and smooth. This is to avoid any injury of delicate mucosa. The outer elastomeric coating is continued well beyond the tip of metal to have a soft malleable tip to protect mucosa. Thus the entire device is made in a beneficial manner and a safe way.

The handle assembly area has a central hollow space to accommodate the nasal septum and columella nasi as described above. This space and circular shape are further reinforced by a U-shaped ring 38 made of metal or rigid elastomeric material which is kept inside the soft outer cover on the frontal aspect of central hollow space. The handle area is a modified part of the end of nasal septal prong on either side. The medially curved extensions of the handle area of nasal septal prongs are held together by the metal axle cum stopper which is inserted into a hole 41 in each of the handle extensions and covered by a flattened part of metal axle cum stopper which bind together the medial handle extensions inside. The spring has more than one loop of coil and each of the free ends are inserted into the inner surface of right handle and other to the left handle.

Essentially, the nasal septal prongs are a smooth rigid structure which is capsular in shape in cross section with smooth curved margins. It has a smooth coating on all surfaces including the one facing air laterally and one touching mucosa medially. It is flat and has a metal or tough elastomeric core inside and has a flat surface on either side. It is to be remembered that slight modifications in the details of the size, material, and modes of construction are well within the scope of invention. The device is ideally made of hypoallergenic external components which are smooth and flexible with a rigid core so as to maintain the nasal septum straight, and nasal airways with almost full dilatation.

In this regard, the air vents in the current invention practically cover most of the anterior nasal mucosa and prevent it from getting blocked and dried up causing discomfort or diseases. It thus improves athletic performance and peaceful sleep, free from snoring or sleep apnea, resulting from blockage of anterior nasal cavity. The elastomeric coating provides safety from injury in case of trauma or unusual movement of the face while sleeping. The metal cores have smooth surfaces and are ideally made of non-breaking material and are further protected by cabling by the soft elastomeric coating. The outer coatings are ideally made of soft silicone gel or soft acrylic or similar material. The air vents are made of relatively rigid, non-breakable but moldable elastomeric medical grade plastic material such as styrene ethylene butylene styrene, SEBS. Apart from material choices, the device upon conception can have several modifications including versions coated with medications or lubrications. They all may fall within the scope of the current invention.

In this regard, more refined versions of the concept are depicted in FIGS. 1 and 2. This embodiment is following the basic principles of the invention of the nasal air flow maintenance and deviation correction device. Notable features of this include slanting inner orifices of the air vents which aid the insertion an easy and a smooth process. It has nasal septal prongs with smooth hypoallergenic elastomeric coated medial and lateral surfaces, a top surface and a bottom surface and a curved smooth tip. The head assembly has a soft malleable and smooth coating covering the metal or rigid frame work of the handle assembly inside. The high-lobed flat cylindroid structure has a large curved area covering the central hollow area and a small knob like area covering the handle area. The soft but strong covering protects the wearer from any possible injury from fall or hit. It also aids the smooth pressing of the handles while inserting and removing the device. A soft elastomeric material like soft acrylic may be an ideal material and shall have suitable coloring either matching the skin of the wearer making it less conspicuous or even colorful. This also helps in a firm grip and decorative purposes. The handle assembly area is to be kept as small as possible to avoid noticeability and not to disrupt sleeping face down. Taking all this into account, the handle assembly may have 10+/−5 mm diameter, flat at upper and lower surfaces.

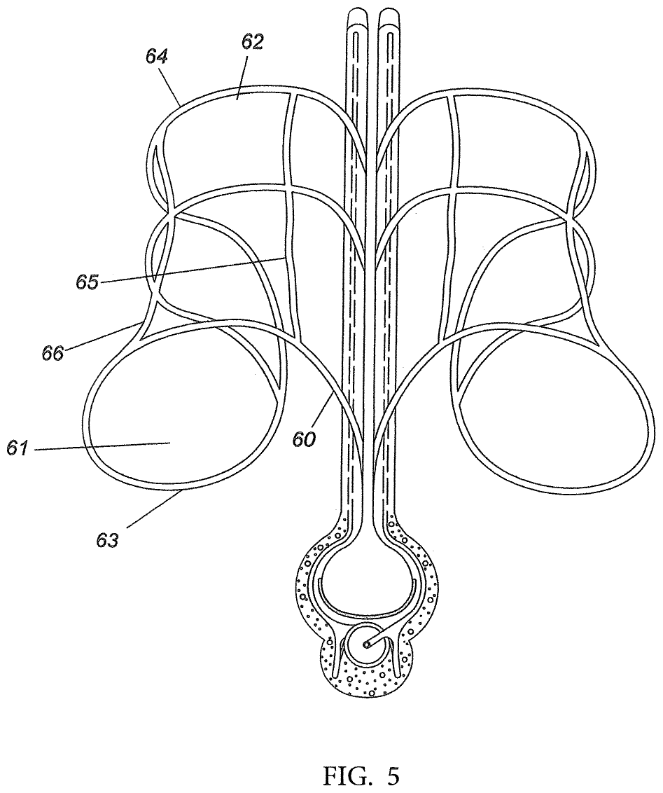

An alternative embodiment of the nasal air flow maintenance and deviation correction device is depicted in FIG. 5. A remarkable feature of this design is a frame work of an elastomeric arch instead of fully cylindrical air vents. The flexible and self-moldable vent guards are fixed to the inner surfaces of nasal septal prongs with two or more attachments 60. It has an anterior frame 63 making a semicircular curvature guarding the anterior part of the nasal cavity and a posterior or distal frame 64 guarding the posterior part of the nasal orifice. These frames are interconnected by more than one cross bars 65. The cross bars are interconnected at many areas so as to maintain integrity and rigidity. This also features a small a relatively small vent guard inflating a large area of the nasal cavity with its curved outline allowing a deep inside position of vent guard leaving a long area in front and behind, not covered by the vent guard. Such a design allows limited contact of the device with nasal mucosa reducing contact-related problems, but maintains the airway diameter. This may be more tolerated, easy to remove, and insert by the wearer.

The unique features of the invention include: 1) a nasal deviation correction device; 2) a nasal air flow regulator which can be kept inside the nasal cavity firmly; 3) a self-moldable air vent; 4) a hygienic and grip enhancing airway maintaining assembly; 5) an ergonomic handle for easy and comfortable use; 6) the aesthetic and safety value of device; and 7) a device solving multitude causes of anterior nasal airway obstruction and covering a large area.

Thus, the preferred embodiment gives enough consideration for safety, benefits, and aesthetic appeal. Several alternative embodiments can be easily derived by removing or slightly modifying one or more of the several features of the invention and those alternative embodiments are well within the scope of present invention.

From a broad viewpoint, the present invention is a nasal air flow maintenance and septal deviation correction device. The device includes a first prong and a similarly configured second prong. Each prong is integrally formed with an upper section and a lower section and an intermediate section there between. Each upper section is linear and of an extended length. Each lower section is arcuate and of a shortened length thereby forming first and second handles. A spring is positioned between the lower sections of the prongs, the spring are coupled to the first and second handles. A first vent is coupled to the upper section of the first prong. A similarly configured second vent is coupled to the to the upper section of the second prong. Each vent has a smaller configuration above and a larger configuration below.

Each embodiment includes a coating on each of the prongs. Further, in each embodiment the intermediate sections of the prongs are generally cylindrical with a cradle there within.

In the primary embodiment, each vent has an axis and the upper end of each vent is circular in a plane perpendicular to the axis. Further, each vent is fabricated of sheet material.

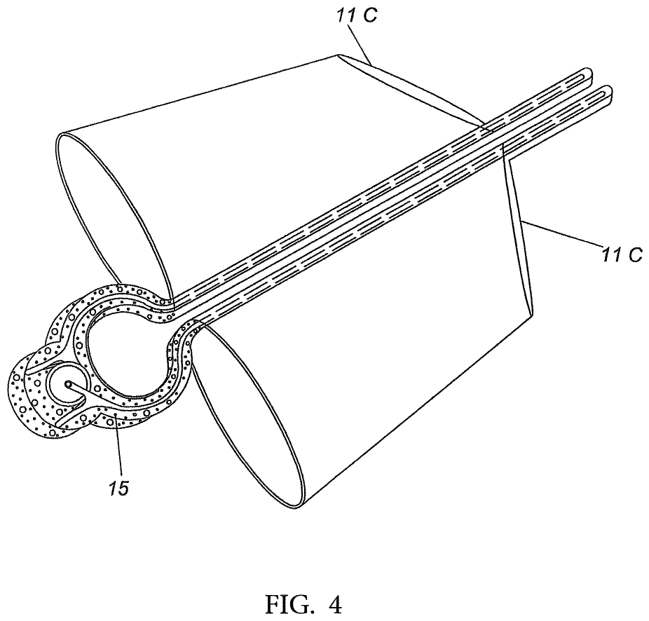

In the embodiment of FIG. 4, each vent has an axis and the upper end 11C. Each is oval and is in a plane at an angle with respect to the axis.

In the embodiment of FIG. 5, each vent is fabricated of wires 60, 61, 62, 63, 64, 65, 66.

From a specific view point, the present invention is a nasal air flow maintenance and septal deviation correction device 10 for maintaining nasal air flow and for correcting sepal deviation of a patient. The maintaining nasal air flow and the correcting sepal deviation are done in a safe, convenient, and economical manner. The device includes a first prong 12A and a similarly configured second prong 12B. Each prong is integrally formed with an upper section 21 and a lower section 22 and an intermediate section 20 there between. Each upper section is linear and of an extended length. Each lower section is arcuate and of a shortened length thereby forming a first handle 30a and a second handle 30B. Each intermediate section is semicircular in configuration. The intermediate sections of the prongs form a generally cylindrical opening 19. Each prong is fabricated of a metallic material.

A coil spring 32 is positioned between the lower sections of the prongs. The coils spring has a first linear end 46 and a second linear end 47 with the first linear end coupled to the first handle and the second linear end coupled to the second handle.

A polymeric coating 15 covers the first and second prongs and the coil spring including the first linear end and the second linear end.

A cradle 38 has a generally U-shaped configuration located within the generally cylindrical opening and positioned upon the polymeric coating. The cradle is fabricated of a rigid polymer.

Lastly provided are a first vent 11A and a similarly configured second vent 11B. Each vent is in a frusto-conical configuration with a smaller circular configuration above and a larger circular configuration below. Each vent is fabricated of a soft polymer sheeting. The first vent is coupled to the upper section of the first prong and the second vent is coupled to the second prong. The coil spring will thus urge the handles apart and the upper sections of the prongs together during use of the device. Further, the handles may be squeezed together to pivot the upper sections of the prongs open.

As to the manner of usage and operation of the present invention, the same should be apparent from the above description. Accordingly, no further discussion relating to the manner of usage and operation will be provided. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention. Therefore, the foregoing is considered as illustrative only of the principles of the invention. Further, since numerous modifications and changes will readily occur to those skilled in the art including an medicated version of the device, it is not desired to limit the invention to the exact construction and operation shown and described, and accordingly, all suitable modifications and equivalents may be resorted to, falling within the scope of the invention.

What is claimed as being new and desired to be protected by Letters Patent of the United States is as follows:

1. A nasal air flow maintenance and septal deviation correction device comprising:
a first prong and a similarly configured second prong, each prong integrally formed with an upper section and a lower section and an intermediate section there between, each upper section being linear and of an extended length, each lower section being arcuate and of a shortened length thereby forming first and second handles;
a spring positioned between the lower sections of the prongs, the spring coupled to the first and second handles; and
a first vent coupled to the to the upper section of the first prong, a similarly configured second vent coupled to the upper section of the second prong, each vent being of a smaller configuration above and a larger configuration below.

2. The device as set forth in claim 1 and further including a coating on each of the prongs.

3. The device as set forth in claim 1 wherein the intermediate sections of the prongs are generally cylindrical with a cradle there within.

4. The system as set forth in claim 1 wherein:
each vent has an axis; and
the upper end of each vent is circular in a plane perpendicular to the axis.

5. The system as set forth in claim 1 wherein each vent is fabricated of sheet material.

6. The system as set forth in claim 1 wherein:
each vent has an axis; and
the upper end of each vent is oval and in a plane at an angle with respect to the axis.

7. The system as set forth in claim 1 wherein each vent is fabricated of wires (60)(61)(62)(63)(64)(65)(66).

8. A nasal air flow maintenance and septal deviation correction device (10) for maintaining nasal air flow and for correcting sepal deviation of a patient, the device comprising, in combination:
a first prong (12A) and a similarly configured second prong (12B), each prong integrally formed with an upper section (21) and a lower section (22) and an intermediate section (20) there between, each upper section being linear and of an extended length, each lower section being arcuate and of a shortened length thereby forming a first handle (30A) and a second handle (30B), each intermediate section being semicircular in configuration, the intermediate sections of the prongs forming a generally cylindrical opening (19), each prong being fabricated of a polymer with limited resilience;
a coil spring (32) positioned between the lower sections of the prongs, the coils spring having a first linear end (46) and a second linear end (47) with the first linear end coupled to the first handle and the second linear end coupled to the second handle;
a polymeric coating (15) covering the first and second prongs and the coil spring including the first linear end and the second linear end;
a cradle (38) having a generally U-shaped configuration located within the generally cylindrical opening and positioned upon the polymeric coating, the cradle being fabricated of a rigid polymer; and
a first vent (11A) and a similarly configured second vent (11B), each vent being in a frusto-conical configuration for with a smaller circular configuration above and a larger circular configuration below, each vent being fabricated of a soft polymer sheeting, the first vent being coupled to the upper section of the first prong and the second vent being coupled to the second prong whereby the coil spring will urge the handles apart and the upper sections of the prongs together during use of the device and whereby the handles may be squeezed together to pivot the upper sections of the prongs open.
